(19) United States
(12) Patent Application Publication
(10) Pub. No.: US 2014/0004601 A1
(43) Pub. Date: Jan. 2, 2014

(54) METHOD OF PURIFYING EXOSOMES

(71) Applicant: Sai Kiang Lim, Singapore (SG)

(72) Inventor: Sai Kiang Lim, Singapore (SG)

(73) Assignee: AGENCY FOR SCIENCE, TECHNOLOGY AND RESEARCH, Singapore (SG)

(21) Appl. No.: 13/994,418

(22) PCT Filed: Dec. 19, 2011
(86) PCT No.: PCT/SG2011/000441

§ 371 Date: Aug. 29, 2013

(30) Foreign Application Priority Data
Dec. 20, 2010 (SG) 201009424-1

(51) Int. Cl. C12N 5/0775

(52) U.S. Cl. CPC C12N 5/0662
USPC 435/317.1

(57) ABSTRACT

We describe a method of purifying a mesenchymal stem cell particle such as an exosome, the method comprising separating the mesenchymal stem cell particle on the basis of its negative charge. The method may comprise (a) providing a composition comprising mesenchymal stem cell particles such as a mesenchymal stem cell conditioned medium (MSC-CM); (b) applying the composition comprising mesenchymal stem cell particles to an ion exchange resin to enable mesenchymal stem cell particles to bind to the ion exchange resin; and (c) eluting bound mesenchymal stem cell particles. The ion exchange resin may comprise an anion exchange resin such as an anion exchange spin column.

### FIELD

The present invention relates to the fields of medicine, cell biology, molecular biology and genetics. This invention relates to the field of medicine.

### BACKGROUND

Exosomes were once thought to be “trash bags” for cells to discard unwanted proteins (Pan et al). However, exosomes are increasingly viewed as having important physiological function particularly in cellular communication.

Exosomes are bi-lipid membrane vesicles of 50-100 ηm that are secreted by many cell types (Thery et al). They belong to a class of secreted cellular products known as microparticles which broadly encompasses all secreted membrane vesicles. Other than exosomes, microparticles include microvesicles (100-1000 ηm), ectosomes (50-200 ηm), membrane particles (50-80 ηm), exosome-like vesicles (20-50 ηm) and apoptotic vesicles (50-500 ηm). The major distinguishing parameter for these different classes of microparticles is their size and the best defined class is the exosomes.

Exosomes have a density in sucrose of 1.10 to 1.19 g/ml, sedimented at 100,000 g, has a cholesterol-rich lipid membrane containing sphingomyelin, ceramide, lipid rafts and exposed phosphatidylserine. The process of exosome biogenesis is complex and involves complex intracellular membrane trafficking and cargo sorting through the biosynthetic and endocytotic pathways. As evidence of this complex biogenesis, the hallmark features of exosomes are markers of the endoplasmic reticulum and the endosomes such as Alix, Tsg101, Rab proteins, etc. Exosomes are stored in multivesicular bodies prior to release via fusion of the multivesicular bodies (MVBs) with the plasma membrane.

Exosomes have been shown to mediate intercellular communication particularly in immune or tumor cells (Fevrier et al; Keller et al; Zitvogel et al; Wolfers, J., et al. et al; Skokos, D., et al. et al; Taylor et al). Recently we extended this function to include tissue repair when we reported that exosomes secreted by human ESC-derived MSCs reduced infarct size by about 50% in a mouse model of myocardial ischemia reperfusion (MI/R) injury (Lai, R. C., et al.; Lai, R. C., et al.). These exosomes were purified as a population of homogenously sized microparticles of about 50-100 ηm in diameter by size exclusion on HPLC and they carry both protein and RNA load (Lai, R. C., et al; Sze, S. K., et al.; Chen, et al).

Efficient purification of biologically active exosomes is crucial to the applications and characterisation of exosomes.

### SUMMARY

According to a 1^{st} aspect of the present invention, we provide a method of purifying a mesenchymal stem cell particle such as an exosome. The method may comprise providing a composition comprising mesenchymal stem cell particles. The method may comprise applying the composition comprising mesenchymal stem cell particles to an ion exchange resin. The method may comprise enabling mesenchymal stem cell particles to bind to the ion exchange resin. The method may comprise eluting bound mesenchymal stem cell particles.

The method may be such that the composition comprising mesenchymal stem cell particles comprises a mesenchymal stem cell conditioned medium (MSC-CM).

The method may be such that the composition comprising mesenchymal stem cell particles is applied an anion exchange resin. The anion exchange resin may comprise an anion exchange spin column.

The method may be such that the composition comprising mesenchymal stem cell particles is applied to the ion exchange spin column at an alkaline pH. The alkaline pH may be for example pH 8.8.

The method may be such that the mesenchymal stem cell particles are eluted at a high salt concentration.

The salt concentration may be 500 μM or more. The salt concentration may be 1 mM or more. The salt concentration may be 2 mM or more. The salt concentration may be 4 mM or more. The salt concentration may be 8 mM or more. The salt concentration may be 16 mM or more. The salt concentration may be 32 mM or more. The salt concentration may be 62.5 mM or more. The salt concentration may be 125 mM or more. The salt concentration may be 250 mM or more. The salt concentration may be 500 mM or more. The salt concentration may be 1M or more. The salt concentration may be 2M NaCl or more.

The method may comprise detecting the alpha subunit of the 20S proteasome in eluted fractions to detect mesenchymal stem cell particles. The method may comprise detecting CD9 in eluted fractions to detect mesenchymal stem cell particles. The method may comprise detecting the alpha subunit of the 20S proteasome and CD9 in eluted fractions to detect mesenchymal stem cell particles. The alpha subunit of the 20S proteasome may be detected by an anti-20S proteasome antibody. The anti-20S proteasome antibody may be one that recognises the alpha subunits. The CD9 may be detected by an anti-CD9 antibody.

The mesenchymal stem cell particle may comprise a particle secreted by a mesenchymal stem cell. The particile may comprise an exosome. The particle may comprise at least one biological property of a mesenchymal stem cell. The biological activity may comprise a biological activity of a mesenchymal stem cell conditioned medium (MSC-CM). The biological activity may comprise cardioprotection.

The mesenchymal stem cell particle may be capable of reducing infarct size for example as assayed in a mouse or pig model of myocardial ischemia and reperfusion injury.

The mesenchymal stem cell particle may be capable of reducing oxidative stress for example as assayed in an in vitro assay of hydrogen peroxide (H₂O₂)-induced cell death.

The mesenchymal stem cell particle may comprise a vesicle such as an exosome. The exosome may comprise at least 70% of proteins in an mesenchymal stem cell conditioned medium (MSC-CM).

The mesenchymal stem cell particle may comprise a complex of molecular weight >100 kDa, for example comprising proteins of <100 kDa.

The mesenchymal stem cell particle may comprise a complex of molecular weight >300 kDa. The mesenchymal stem cell particle may comprise proteins of <300 kDa.

The mesenchymal stem cell particle may comprise a complex of molecular weight >1000 kDa.

The mesenchymal stem cell particle may have a size of between 2 nm and 200 nm. The mesenchymal stem cell particle may have a size of between 50 nm and 150 nm.

[symbol]

The size may be as determined by filtration against a 0.2 μM filter and concentration against a membrane with a molecular weight cut-off of 10 kDa. The size may be as determined by electron microscopy.

The mesenchymal stem cell particle may comprise a hydrodynamic radius of below 100 nm.

The mesenchymal stem cell particle may comprise a hydrodynamic radius of between about 30 nm and about 70 nm.

The mesenchymal stem cell particle may comprise a hydrodynamic radius of between about 40 nm and about 60 nm.

The mesenchymal stem cell particle may comprise a hydrodynamic radius of between about 45 nm and about 55 nm.

The mesenchymal stem cell particle may comprise a hydrodynamic radius of about 50 nm. The hydrodynamic radius may be as determined by laser diffraction or dynamic light scattering.

The mesenchymal stem cell particle may comprise a lipid selected from the group consisting of: phospholipid, phosphatidyl serine, phosphatidyl inositol, phosphatidyl choline, shingomyelin, ceramides, glycolipid, cerebroside, steroids, cholesterol. The cholesterol-phospholipid ratio may be greater than 0.3-0.4 (mol/mol) .

The mesenchymal stem cell particle may comprise a lipid raft.

The mesenchymal stem cell particle may be insoluble in non-ionic detergent. The non-ionic detergent may comprise Triton-X100.

The mesenchymal stem cell particle may be such that proteins of the molecular weights specified above substantially remain in the complexes of the molecular weights as specified, when the particle is treated with a non-ionic detergent.

The mesenchymal stem cell particle may be sensitive to cyclodextrin. The mesenchymal stem cell particle may be sensitive to 20 mM cyclodextrin. Treatment with cyclodextrin may cause substantial dissolution of the complexes specified above.

The mesenchymal stem cell particle may comprise ribonucleic acid (RNA). The particle may have an absorbance ratio of 1.9 (260:280 nm).

The mesenchymal stem cell particle may comprise a surface antigen selected from the group consisting of: CD9, CD109 and thy-1.

There is provided, according to a 2^{nd} aspect of the present invention, a method of producing a mesenchymal stem cell particle such as an exosome. The method may comprise separating the particle from other components of a composition such as a mesenchymal stem cell conditioned medium (MSC-CM) based on charge.

The method may comprise anion exchange chromatography.

The method may comprise the use of an anion exchange spin column.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-

[symbol]

### BRIEF DESCRIPTION OF THE FIGURES

FIG. 1 is a diagram showing fractionation of MSC conditioned medium by anion exchange chromatography.

FIG. 2 is a diagram showing fractionation of MSC conditioned medium by cation exchange chromatography.

### DETAILED DESCRIPTION

Therapeutic exosomes have previously been separated from MSC secretions by size exclusion chromatography.

This disclosure describes a novel method for the purification of exosomes based on their negative charge. In particular, the present technology describes the enrichment of exosomes by ion exchange chromatography, such as ion exchange spin column chromatography, in particular, anion exchange chromatography.

Our invention is based on the surprising demonstration that exosomes from MSCs are negatively charged. As such, they have a low or acidic isoelectric point, pI. This is wholly unexpected as previous reports have shown that exosomes from tumours are positively charged (i.e., with a high or basic pI). Thus, Graner et al (2009) isolated exosomes from brain tumour cells and determined their biochemical properties. They demonstrate using isoelectric focusing that such exosomes focused to the far basic end of the pH gradient, indicating that they have high pIs and are accordingly positively charged.

Our discovery thereforeenables a novel method to purify or isolate exosomes from mesenchymal stem cells, by use of their negative charge. Thus, with our discovery, it is possible to provide a method of separating exosomes from other components by use of, for example, anion exchange resins.

Our methodology for exosome purification is highly scalable in terms of time and quantity. This methodology allows different exosomes to be distinguished and purified on the basis of charges. This methodology could potentially be adapted for small scale spin columns as well as high resolution and/or high capacity HPLC or FPLC systems.

### Purifying Mesenchymal Stem Cell Particles

The mesenchymal stem cell particle may be purified, using the methods described in this document, from any suitable source.

By “purifying” a particle (such as a mesenchymal stem cell exosome) from a composition comprising the particle and one or more contaminants is meant increasing the degree of purity of the particle in the composition by removing (completely or partially) at least one contaminant from the composition. A “purification step” may be part of an overall purification process resulting in a “homogeneous” composition. “Homogeneous” is used herein to refer to a composition comprising at least about 70% by weight of the particle of interest, based on total weight of the composition, such as at least about 80% by weight, such as at least about 90% by weight, such as at least about 95% by weight.

Examples of suitable sources include conditioned cell culture medium such as a Mesenchymal Stem Cell Conditioned Medium (MSC-CM). Accordingly, our methods may involve isolating the exosome from a mesenchymal stem cell (MSC) or from an mesenchymal stem cell conditioned medium (MSC-CM), on the basis of its negative charge.

The mesenchymal stem cell may be produced by any suitable process, as known in the art, as described in further detail below. An example of such a process comprises obtaining a cell by dispersing a embryonic stem (ES) cell colony. The cell, or a descendent thereof, may be propagated in the absence of co-culture in a serum free medium comprising FGF2.

Mesenchymal stem cell conditioned medium may be obtained by culturing a mesenchymal stem cell (MSC), a descendent thereof or a cell line derived therefrom in a cell culture medium and isolating the cell culture medium.

In general, we describe a method for separating a particle from other entities in a sample. The other entities may comprise things which are not of interest, and from which separation of the particle is desired. We refer to these for convenience as “contaminants”. The particle may comprise a particle from a stem cell, such as secreted by a stem cell. The stem cell may comprise a mesenchymal stem cell. The particle may comprise a vesicle, or microvesicle, or an exosome.

The method may comprise purification, isolation or separation of a mesenchymal stem mesenchymal stem cell particle/exosome from its environment or contaminants by means of charge.

For example, we describe a method which comprises loading a composition comprising mesenchymal stem cell particles onto an ion exchange resin. The ion exchange resin may comprise an anion exchange resin. The ion exchange resin may be in the form of a spin column. The composition may be loaded with an equilibration buffer. The ion exchange resin may be washed with a wash buffer. The particles may be eluted by a salt gradient.

### Separating Mesenchymal Stem Cell Exosomes by Charge

According to the methods and compositions described here, ion exchange chromatography may be used to separate and/or purify exosomes from mesenchymal stem cells. The separation may be in a small (analytical) or large (preparative) scale.

Ion-exchange chromatography (or ion chromatography) is a process that allows the separation of ions and polar molecules based on their charge. It can be used for almost any kind of charged molecule including large proteins, small nucleotides and amino acids.

Ion-exchange chromatography retains analyte molecules on the column based on coulombic (ionic) interactions. The stationary phase surface displays ionic functional groups (R—X) that interact with analyte ions of opposite charge. In the present example, anion exchange chromatography may be employed to purify or separate mesenchymal stem cell exosomes.

Anion exchange chromatography retains anions using positively charged functional group on the resin, which binds to negatively charged exosomes from mesenchymal stem cells.

In the methods described here using ion exchange chromatography, a sample comprising mesenchymal stem cell particles (such as exosomes) is introduced, either manually or with an autosampler, into a sample loop of known volume. A buffered aqueous solution known as the mobile phase carries the sample from the loop onto a column that contains some form of stationary phase material.

To optimize binding of all charged molecules, the mobile phase is generally a low to medium conductivity (i.e., low to medium salt concentration) solution. The adsorption of the charged ionic groups in the sample molecule and in the functional ligand on the support. The strength of the interaction is determined by the number and location of the charges on the molecule and on the functional group. By increasing the salt concentration (generally by using a linear salt gradient) the molecules with the weakest ionic interactions start to elute from the column first. Molecules that have a stronger ionic interaction require a higher salt concentration and elute later in the gradient. The binding capacities of ion exchange resins are generally quite high. This is of major importance in process scale chromatography, but is not critical for analytical scale separations.

The stationary phase material may comprise a resin or gel matrix consisting of for example agarose or cellulose beads with covalently bonded charged functional groups. The target analytes (anions in the case of exosomes from mesenchymal stem cells) are retained on the stationary phase.

As a rule, the pH of the mobile phase buffer must be between the pI (isoelectric point) or pKa (acid dissociation constant) of the charged particle and the pKa of the charged group on the solid support. For example, in anion exchange chromatography a molecule with a pI of 6.8 may be run in a mobile phase buffer at pH 8.0 when the pKa of the solid support is 10.3.

The composition comprising mesenchymal stem cell particles may be applied to the ion exchange spin column at an alkaline pH. The pH may be pH 7.4 or higher, pH 7.5 or higher, pH 7.6 or higher, pH 7.7 or higher, pH 7.8 or higher, pH 7.9 or higher, pH 8.0 or higher, pH 8.1 or higher, pH 8.2 or higher, pH 8.3 or higher, pH 8.4 or higher, pH 8.5 or higher, pH 8.6 or higher, pH 8.7 or higher, pH 8.8 or higher, pH 8.9 or higher, pH 9.0 or higher, pH 9.1 or higher, pH 9.2 or higher, pH 9.3 or higher, pH 9.4 or higher, pH 9.5 or higher, pH 9.6 or higher, pH 9.7 or higher, pH 9.8 or higher, pH 9.9 or higher or pH 10.0 or higher. For example, the pH may be about pH 8.8.

### Elution and Detection

The bound exosomes from mesenchymal stem cells may be eluted by increasing the concentration of a similarly charged species that will displace the analyte ions from the stationary phase. The bound exosomes may be eluted by applying a gradient of linearly increasing salt concentration. Alternatively, a step gradient may be employed. This requires less complicated equipment and can be very effective to elute different fractions if the

[symbol]

For example, bound mesenchymal stem cell exosomes may be displaced by the addition of negatively charged ions such as chloride ions.

Thus, bound mesenchymal stem cell particles may be eluted at a high salt concentration. The salt may comprise any suitable chloride salt. The salt may comprise for example sodium chloride, i.e., NaCl. The salt concentration may be 500 μM or more, 1 mM or more, 2 mM or more, 4 mM or more, 8 mM or more, 16 mM or more, 32 mM or more, 62.5 mM or more, 125 mM or more, 250 mM or more, 500 mM or more, 1M or more or 2M NaCl or more.

Changes in pH may also be used to affect a separation. In anion exchange chromatography, lowering the pH of the mobile phase buffer will cause the particle to become more protonated and hence more positively (and less negatively) charged. The result is that the protein no longer can form a ionic interaction with the positively charged solid support which causes the molecule to elute from the column.

The analytes of interest may be detected by any suitable means, typically by conductivity or UV/Visible light absorbance.

Alternatively or in addition, polypeptides diagnostic of the analyte of interest (here exosomes from mesenchymal stem cells) may be detected by, e.g., immunochemistry.

Thus, for example, the method may comprise detecting any antigen known to be present on mesenchymal stem cell exosomes. Such antigens are for example described in detail in International Patent Application WO 2009/105044.

An example of an antigen is the alpha subunit of the 20S proteasome. A further example is CD9. Thus, the methods described here may include detection of either the alpha subunit of the 20S proteasome, or CD9, or both, in eluted fractions as a means to detect fractions comprising mesenchymal stem cell particles. The alpha subunit of the 20S proteasome or CD9, or both, may be detected by any suitable means. The alpha subunit of the 20S proteasome may for example be detected by an anti-20S proteasome antibody that recognises the alpha subunits. The CD9 may be detected by an anti-CD9 antibody, or both.

### Other Purification Methods

The methods described here may be combined with other known methods of purifying example separating the exosome from non-associated components based on any property of the exosome. For example, the exosome may be isolated based on molecular weight, size, shape, composition or biological activity.

The conditioned medium may be filtered or concentrated or both during, prior to or subsequent to separation, including the separation methods described here which rely on the negative charge of the exosome. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the exosome may be used to track its activity during fractionation of the mesenchymal stem cell conditioned medium (MSC-CM). As an example, light scattering, refractive index, dynamic light scattering or UV-visible detectors may be used to follow the exosome. For example, a therapeutic activity such as cardioprotective activity may be used to track the activity during fractionation.

### Example Protocol

The following paragraphs provide a specific example of how a mesenchymal stem cell exosome may be obtained.

A mesenchymal stem cell exosome may be produced by culturing mesenchymal stem cells in a medium to condition it. The mesenchymal stem cells may comprise HuES9.E1 cells. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-

[symbol]

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more.

The conditioned medium may then be subjected to anion exchange chromatography, as described in the Examples.

In addition to detecting the alpha subunit of the 20S proteasome and CD9, UV absorbance such as at 220 nm may also be used to track the progress of elution. Fractions may be examined for dynamic light scattering (DLS) using a quasi-elastic light scattering (QELS) detector.

Fractions which are found to exhibit dynamic light scattering may be retained. In addition to anion exchange chromatography, the conditioned medium may be separated by size exclusion, as an additional step. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic.

For example, a fraction which is produced by the general method as described above, and which elutes with a retention time of 11-13 minutes, such as 12 minutes, is found to exhibit dynamic light scattering. The rₕ of exosomes in this peak is about 45-55 nm. Such fractions comprise mesenchymal stem cell exosomes.

### Ion Exchange

As the term is used in this document, “ion exchange material” refers to a solid phase that is negatively charged (i.e. a cation exchange resin) or positively charged (i.e. an anion exchange resin). The charge may be provided by attaching one or more charged ligands to the solid phase, e.g. by covalent linking. Alternatively, or in addition, the charge may be an inherent property of the solid phase (e.g. as is the case for silica, which has an overall negative charge).

An ion-exchange resin or ion-exchange polymer may comprise an insoluble matrix (or support structure) normally in the form of small (1-2 mm diameter) beads. It is usually fabricated from an organic polymer substrate. The material has highly developed structure of pores on the surface of which are sites with easily trapped and released ions. The trapping of ions takes place only with simultaneous releasing of other ions; thus the process is called ion-exchange. There are multiple different types of ion-exchange resin which are fabricated to selectively prefer one or several different types of ions.

Most typical ion-exchange resins are based on crosslinked polystyrene. The required active groups can be introduced after polymerization, or substituted monomers can be used. For example, the crosslinking is often achieved by adding 0.5-25% of divinylbenzene to styrene at the polymerization process. Non-crosslinked polymers are used only rarely because they are less stable. Crosslinking decreases ion- exchange capacity of the resin and prolongs the time needed to accomplish the ion exchange processes. Particle size also influences the resin parameters; smaller particles have larger outer surface, but cause larger head loss in the column processes.

There are four main types differing in their functional groups:
strongly acidic (typically, sulfonic acid groups, e.g. sodium polystyrene sulfonate or polyAMPS)
strongly basic, (quaternary amino groups, for example, trimethylammonium groups, e.g. polyAPTAC)
weakly acidic (mostly, carboxylic acid groups)
weakly basic (primary, secondary, and/or ternary amino groups, e.g. polyethylene amine)

Examples of anion exchange resins include, but are not limited to: Amberlite® IRA-67 free base gel form, 16-50 mesh (wet) (A9960), Benzoylated Naphthoylated DEAE-Cellulose medium (B6385), Cholestyramine resin (C4650), DEAE-Cellulose preswollen, microgranular (D3764), DEAE-Cellulose Fast Flow, fibers (D6418), DEAE-Cellulose fibers (D0909), DEAE-Sephadex® (A50120), DEAE-Sephadex® A-25 chloride form (A25120), DEAE-Sepharose® CL-6B (DCL6B 100), DEAE-Sepharose® Fast Flow (DFF 100), DEAE-aqueous ethanol suspension, 40-80 μm (D2540), Diethylaminoethyl-Sephacel® aqueous ethanol suspension, 40-160 μm (wet), exclusion limit ˜1,000,000 Da (16505), QAE Sephadex® A-25 chloride form (Q25120), QAE Sephadex® A-50 chloride form (Q50120), Q Sepharose® Fast Flow preswollen, 45-165 μm (wet), exclusion limit ˜4,000,000 Da (Q1126), Q Sepharose® High Performance preswollen, 24-44 μm (wet), average exclusion limit ˜4,000,000 Da (Q1754), TEAE cellulose (T9658) (Sigma-Aldrich catalogue numbers in brackets; Sigma-Aldrich, St Louis, Mo., USA).

### Ion Exchange Resins

Ion exchange resins of interest to the methods and compositions described here include anion exchange resins, which comprise basic charges.

“Solid phase” means a non-aqueous matrix to which one or more charged ligands can adhere. The solid phase may be a purification column, a discontinuous phase of discrete particles, a membrane, or filter etc. Examples of materials for forming the solid phase include polysaccharides (such as agarose and cellulose) and other mechanically stable matrices such as silica (e.g. controlled pore glass), poly(styrenedivinyl)benzene, polyacrylamide, ceramic particles and derivatives of any of the above.

The term “anion exchange resin” is used in this document to refer to a solid phase which is positively charged, e.g. having one or more positively charged ligands, such as quaternary amino groups, attached thereto. Commercially available anion exchange resins include DEAE cellulose, QAE SEPHADEX and FAST Q SEPHAROSE (Pharmacia).

A “buffer” is a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., Ed. Calbiochem Corporation (1975). In one embodiment, the buffer has a pH in the range from about 5 to about 7 (e.g. as in Example 1 below). Examples of buffers that will control the pH in this range include MES, MOPS, MOPSO, phosphate, acetate, citrate, succinate, and ammonium buffers, as well as combinations of these. The buffers used in the disclosed methods typically also comprise a salt, such as NaCL, KCl or NaHOAc.

An “equilibration buffer” may be used to equilibrate the ion exchange resin. The equilibration buffer may also be used to load the composition comprising the mesenchymal stem cell particle (such as an exosome) and one or more contaminants onto the ion exchange resin. The equilibration buffer preferably has a conductivity and/or pH such that the mesenchymal stem cell particle (such as an exosome) is bound to the ion exchange resin.

A “wash buffer” is used in this document to refer to the buffer that is passed over the ion exchange resin following loading and prior to elution of the mesenchymal stem cell particles (such as exosomes). The wash buffer may serve to elute one or more contaminants from the ion exchange resin. The conductivity and/or pH of the wash buffer is/are such that the contaminants are eluted from the ion exchange resin, but not significant amounts of the mesenchymal stem cell particles (such as exosomes). The “wash buffer” preferably comprises a mixture of equilibration buffer and elution buffer, and can thus be described by the percentage of elution buffer that it comprises in a given volume.

An “elution buffer” may be used to elute the bound mesenchymal stem cell particles (such as exosomes) from the solid phase. The conductivity and/or pH of the elution buffer is/are such that the mesenchymal stem cell particle (such as exosome) is eluted from the ion exchange resin.

A “regeneration buffer” may be used to regenerate the ion exchange resin such that it can be re-used. The regeneration buffer has a conductivity and/or pH as required to remove substantially all contaminants and the mesenchymal stem cell particles (such as exosomes) from the ion exchange resin.

### Ion Exchange Spin Columns

As a specific example, the ion exchange resin may be provided in the form of an ion exchange spin column.

Ion exchange spin columns use the membrane-adsorber technology as a chromatographic matrix to fractionate proteins based on their charge differences. Ion exchange columns use membrane adsorbers that have a highly porous structure with pores larger than 3000 nm, providing proteins easy access to the membrane's charged ligands. Therefore, adsorptive membranes maintain high efficiencies at high-flow rates and when fractionating large biomolecules with small diffusivities.

Their membrane-based spin format eliminates column packing, while the centrifugal format enables processing of multiple samples in parallel. Membrane adsorber spin columns do not crack or run dry and small membrane adsorber bed volumes make working with low buffer volumes possible, leading to concentrated elution fractions.

Sample is loaded on to the spin column, and spun. The analyte (e.g., mesenchymal stem cell particles such as exosomes) is bound to the membrane. The spin column is washed with wash buffer and then elution buffer is applied. The column is spun to elute the analyte.

Ion exchange spin columns are available from a variety of manufacturers, including Pierce (Thermo Fisher Scientific, Pierce Protein Research Products, Rockford, Ill., USA). Examples include Pierce Strong Cation Exchange Spin Column, Mini (Pierce catalogue number 90008), which comprise polypropylene microcentrifuge columns with bottom ion exchange filter membrane; microcentrifuge collection tubes; Pierce Strong Cation Exchange Spin Column, Maxi (Pierce catalogue number 90009), which comprise polypropylene centrifuge columns with bottom ion exchange filter membrane; 50 mL centrifuge collection tubes; Pierce Strong Anion Exchange Spin Column, Mini (Pierce catalogue number 90010), which comprise polypropylene microcentrifuge columns with bottom ion exchange filter membrane; Microcentrifuge collection tubes; Pierce Strong Anion Exchange Spin Column, Maxi (Pierce catalogue number 90011), which comprise polypropylene centrifuge columns with bottom ion exchange filter membrane; 50 mL centrifuge collection tubes.

Ion exchange spin columns are also available under the Vivapure Mini Spin Columns and Vivapure Maxi Spin Columns brand names, obtainable from Sartorius (Goettingen, Germany). These include Vivapure Q Mini M (catalogue number VS-IX01QM24); Vivapure Q Mini H (catalogue number VS-IX01QH24); Vivapure Q Maxi M (catalogue number VS-IX20QM08); and Vivapure Q Maxi H (catalogue number VS-IX20QH08).

### Exosomes

Exosomes are small membrane vesicles formed in late endocytic compartments (multivesicular bodies) first described to be secreted by reticulocytes in 1983 and subsequently found to be secreted by many cells types including various haematopoietic cells, tumours of haematopoietic or non-haematopoietic origin and epithelial cells. They are distinct entities from the more recently described ‘ribonuclease complex’ also named exosome.

Exosomes may be defined by a number of morphological and biochemical parameters. Accordingly, the exosome described here may comprise one or more of these morphological or biochemical parameters.

Exosomes are classically defined as “saucer-like” vesicles or a flattened sphere limited by a lipid bilayer with diameters of 40-100 nm and are formed by inward budding of the endosomal membrane. Like all lipid vesicles and unlike protein aggregates or nucleosomal fragments that are released by apoptotic cells, exosomes have a density of ˜1.13-1.19 g/ml and float on sucrose gradients. Exosomes are enriched in cholesterol and sphingomyelin, and lipid raft markers such as GM1, GM3, flotillin and the src protein kinase Lyn suggesting that their membranes are enriched in lipid rafts.

The molecular composition of exosomes from different cell types and of different species has been examined. In general, exosomes contain ubiquitous proteins that appear to be common to all exosomes and proteins that are cell-type specific. Also, proteins in exosomes from the same cell-type but of different species are highly conserved. The ubiquitous exosome-associated proteins include cytosolic proteins found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. The tetraspannins are highly enriched in exosomes and are known to be involved in the organization of large molecular complexes and membrane subdomains.

Examples of cell-type specific proteins in exosomes are MHC class II molecules in exosomes from MHC class II-expressing cells, CD86 in dendritic cell-derived exosomes, T-cell receptors on T-cell-derived exosomes etc. Notably, exosomes do not contain proteins of nuclear, mitochondrial, endoplasmic-reticulum or Golgi-apparatus origin. Also, highly abundant plasma membrane proteins are absent in exosomes suggesting that they are not simply fragments of the plasma membrane. Many of the reported ubiquitous exosome-associated proteins are also present in the proteomic profile of the hESC-MSC secretion.

Exosomes are also known to contain mRNA and microRNA, which can be delivered to another cell, and can be functional in this new location. The physiological functions of [symbol] proteins, mediate tramission of infectious particles such as prions and viruses, induce complement resistance, facilitate immune cell-cell communication and transmit cell signaling. Exosomes have been used in immunotherapy for treatment of cancer.

### Exosome Molecular Weight

The exosome may have a molecular weight of greater than 100 kDa. It may have a molecular weight of greater than 500 kDa. For example, it may have a molecular weight of greater than 1000 kDa.

The molecular weight may be determined by various means. In principle, the molecular weight may be determined by size fractionation and filtration through a membrane with the relevant molecular weight cut-off The exosome size may then be determined by tracking segregation of component proteins with SDS-PAGE or by a biological assay.

### Assay of Molecular Weight by SDS-PAGE

The exosome may have a molecular weight of greater than 100 kDa. For example, the exosome may be such that most proteins of the exosome with less than 100 kDa molecular weight segregate into the greater than 100 kDa molecular weight retentate fraction, when subject to filtration. Similarly, when subjected to filtration with a membrane with a 500 kDa cut off, most proteins of the exosome with less than 500 kDa molecular weight may segregate into the greater than 500 kDa molecular weight retentate fraction. This indicates that the exosome may have a molecular weight of more than 500 kDa.

### Assay of Molecular Weight by Biological Activity

The exosome may have a molecular weight of more than 1000 kDa. For example, the exosome may be such that when subject to filtration with a membrane with a molecular weight cutoff of 1000 kDa, the relevant biological activity substantially or predominantly remains in the retentate fraction. Alternatively or in addition, biological activity may be absent in the filtrate fraction. The biological activity may comprise any of the biological activities of the exosome described elsewhere in this document.

### Assay of Molecular Weight by Infarct Size

For example, the biological activity may comprise reduction of infarct size, as assayed in any suitable model of myocardia ischemia and reperfusion injury. For example, the biological activity may be assayed in a mouse or pig model, as described in WO 2009/105044.

In summary, myocardial ischemia is induced by 30 minutes left coronary artery (LCA) occlusion by suture ligation and reperfusion is initiated by removal of suture. Mice are treated with liquid containing the exosomes (such as unfractionated MSC-CM), filtrate (such as <100 or 1,000 kD fraction), retentate (such as >1000 kD retentate) or saline intravenously via the tail vein, 5 minutes before reperfusion. 24 hours later, the hearts are excised. Before excision, the Area At Risk (AAR) is determined by religating the LCA and then perfusing Evans blue through the aorta.

AAR is defined as the area not stained by the dye and is expressed as a percentage of the left ventricular wall area. Infarct size is assessed 24 hours later using Evans blue and TTC. Where the relative infarct size is significantly reduced in animals treated with mesenchymal stem cell conditioned medium (MSC-CM) and the retentate (such as a >1000 kD) fraction when compared to saline, this indicates that the exosome has a molecular weight which is higher than the relevant cutoff of the membrane (e.g., greater than 1000 kDa).

### Exosome Size

The exosome may have a size of greater than 2 nm. The exosome may have a size of greater than 5 nm, 10 nm, 20 nm, 30 nm, 40 nm or 50 nm. The exosome may have a size of greater than 100 nm, such as greater than 150 nm. The exosome may have a size of substantially 200 nm or greater.

The exosome may have a range of sizes, such as between 2 nm to 20 nm, 2 nm to 50 nm, 2 nm to 100 nm, 2 nm to 150 nm or 2 nm to 200 nm. The exosome may have a size between 20 nm to 50 nm, 20 nm to 100 nm, 20 nm to 150 nm or 20 nm to 200 nm. The exosome may have a size between 50 nm to 100 ηm, 50nm to 150 nm or 50 nm to 200 nm. The exosome may have a size between 100 nm to 150 nm or 100 nm to 200 nm. The exosome may have a size between 150 nm to 200 nm.

The size may be determined by various means. In principle, the size may be determined by size fractionation and filtration through a membrane with the relevant size cut-off. The exosome size may then be determined by tracking segregation of component proteins with SDS-PAGE or by a biological assay.

The size may also be determined by electron microscopy.

The size may comprise a hydrodynamic radius. The hydrodynamic radius of the exosome may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the exosome may be determined by any suitable means, for example, laser diffraction or dynamic light scattering. An example of a dynamic light scattering method to determine hydrodynamic radius is described in WO 2009/105044.

### Obtaining Mesenchymal Stem Cells (MSC)

Mesenchymal stem cell particles such as exosomes may be isolated or produced, using the methods described here, from mesenchymal stem cell conditioned medium (MSC-CM).

MSCs suitable for use in the production of conditioned media and exosomes may be made by any method known in the art.

In particular, MSCs may be made by propagating a cell obtained by dispersing a embryonic stem (ES) cell colony, or a descendent thereof, in the absence of co-culture in a serum free medium comprising FGF2. This is described in detail in the sections below.

Methods of obtaining mesenchymal stem cells (MSC) or MSC-like cells from hESCs may involve either transfection of a human telomerase reverse transcriptase (hTERT) gene into differentiating hESCs (Xu et al., 2004) or coculture with mouse OP9 cell line (Barberi et al., 2005). The use of exogenous genetic material and mouse cells in these derivation protocols introduces unacceptable risks of tumorigenicity or infection of xenozootic infectious agents.

The exosomes may therefore be made from MSCs derived by the use of a clinically relevant and reproducible protocol for isolating similar or identical (such as homogenous) MSC populations from differentiating hESCs. In general, the method comprises dispersing a embryonic stem (ES) cell colony into cells. The cells are then plated out and propagated. The cells are propagated in the absence of co-culture in a serum free medium comprising fibroblast growth factor 2 (FGF2), in order to obtain mesenchymal stem cells (MSCs).

Thus, the protocol does not require serum, use of mouse cells or genetic manipulations and requires less manipulations and time, and is therefore highly scalable. The protocol may third one, Hes-3. Human ES cell derived MSCs (hESC-MSCs) obtained by the methods and compositions described here are remarkably similar to bone-marrow derived MSCs (BM-MSCs).

The embryonic stem cell culture may comprise a human embryonic stem cell (hESC) culture.

In a one embodiment, a method of generating mesenchymal stem cells (MSC) comprises trypsinizing and propagating hESCs without feeder support in media supplemented with FGF2 and optionally PDGF AB before sorting for CD105+CD24-cells.

The method may comprise sorting for CD105+, CD24-cells from trypsinized hESCs one week after feeder-free propagation in a media supplemented with FGF2 and optionally PDGF AB will generate to generate a hESC-MSC cell culture in which at least some, such as substantially all, or all cells are similar or identical (such as homogenous) to each other

The MSCs produced by this method may be used to produce mesenchymal stem cell conditioned medium (MSC-CM), from which the exosomes may be isolated.

### Disaggregating Embryonic Stem Cell Colonies

One method of producing mesenchymal stem cells may comprise dispersing or disaggregating an embryonic stem cell colony into cells.

The embryonic stem cell colony may comprise a huES9 colony (Cowan C A, Klimanskaya I, McMahon J, Atienza J, Witmyer J, et al. (2004) Derivation of embryonic stem-cell lines from human blastocysts. N Engl J Med 350: 1353-1356) or a H1 ESC colony (Thomson J A, Itskovitz-Eldor J, Shapiro S S, Waknitz M A, Swiergiel J J, et al. (1998) Embryonic Stem Cell Lines Derived from Human Blastocysts. Science 282: 1145-1147.).

The cells in the colony may be disaggregated or dispersed to a substantial extent, i.e., at least into clumps. The colony may be disaggregated or dispersed to the extent that all the cells in the colony are single, i.e., the colony is completely disaggregated.

The disaggregation may be achieved with a dispersing agent.

The dispersing agent may be anything that is capable of detaching at least some embryonic stem cells in a colony from each other. The dispersing agent may comprise a reagent which disrupts the adhesion between cells in a colony, or between cells and a substrate, or both. The dispersing agent may comprise a protease.

The dispersing agent may comprise trypsin. The treatment with trypsin may last for example for 3 minutes or thereabouts at 37 degrees C. The cells may then be neutralised, centrifuged and resuspended in medium before plating out.

The method may comprise dispersing a confluent plate of human embryonic stem cells with trypsin and plating the cells out.

The disaggregation may comprise at least some of the following sequence of steps: aspiration, rinsing, trypsinization, incubation, dislodging, quenching , re-seeding and aliquoting. The following protocol is adapted from the Hedrick Lab, UC San Diego (http://hedricklab.ucsd.edu/Protocol/COSCell.html).

In the aspiration step, the media is aspirated or generally removed from the vessel, such as a flask. In the rinsing step, the cells are rinsed with a volume, for example 5-10 mls, of a buffered medium, which is may be free from Ca²⁺ and Mg²⁺. For example, the cells may be rinsed with calcium and magnesium free PBS. In the trypsinization step, an amount of dispersing agent in buffer is added to the vessel, and the vessel rolled to coat the growing surface with the dispersing agent solution. For example, 1 ml of trypsin in Hank's BSS may be added to a flask.

In the incubation step, the cells are left for some time at a maintained temperature. For example, the cells may be left at 37° C. for a few minutes (e.g., 2 to 5 minutes). In the dislodging step, the cells may be dislodged by mechanical action, for example by scraping or by whacking the side of the vessel with a hand. The cells should come off in sheets and slide down the surface.

In the quenching step, a volume of medium is added to the flask. The medium may comprise a neutralising agent to stop the action of the dispersing agent. For example, if the dispersing agent is a protease such as trypsin, the medium may contain a protein, such as a serum protein, which will mop up the activity of the protease. In a particular example, 3 ml of serum containing cell culture medium is added to the flask to make up a total of 4 mls. The cells may be pipetted to dislodge or disperse the cells.

In the re-seeding step, the cells are re-seeded into fresh culture vessels and fresh medium added. A number of re-seedings may be made at different split ratios. For example, the cells may be reseeded at 1/15 dilution and ⅕ dilution. In a particular example, the cells may be re-seeded by adding 1 drop of cells into a 25 cm² flask and 3 drops into another to re-seed the culture, and 7-8 mls media is then added to each to provide for 1/15 dilution and ⅕ dilution from for example a 75 cm² flask. In the aliquoting step, the cells may be aliquoted into new dishes or whatever split ratio is desired, and media added.

In a specific embodiment, the method includes the following steps: human ES cells are first grown suspended in non-adherent manner to form embryoid bodies (EBs). 5-10 day old EBs are then trypsinized before plating as adherent cells on gelatine coated tissue culture plates.

### Maintenance as Cell Culture

The disaggregated cells may be plated and maintained as a cell culture.

The cells may be plated onto a culture vessel or substrate such as a gelatinized plate. Crucially, the cells are grown and propagated without the presence of co-culture, e.g., in the absence of feeder cells.

The cells in the cell culture may be grown in a serum-free medium which is supplemented by one or more growth factors such as fibroblast growth factor 2 (FGF2) and optionally platelet-derived growth factor AB (PDGF AB), at for example 5 ng/ml. The cells in the cell culture may be split or subcultured 1:4 when confluent, by treatment with trypsin, washing and replating.

### Absence of Co-Culture

The cells may be cultured in the absence of co-culture. The term “co-culture” refers to a mixture of two or more different kinds of cells that are grown together, for example, stromal feeder cells.

Thus, in typical ES cell culture, the inner surface of the culture dish is usually coated with a feeder layer of mouse embryonic skin cells that have been treated so they will not divide. The feeder layer provides an adherent surface to enable the ES cells to attach and grow. In addition, the feeder cells release nutrients into the culture medium which are required for ES cell growth. In the methods and compositions described here, the ES and MSC cells may be cultured in the absence of such co-culture.

The cells may be cultured as a monolayer or in the absence of feeder cells. The embryonic stem cells may be cultured in the absence of feeder cells to establish mesenchymal stem cells (MSC).

The dissociated or disaggregated embryonic stem cells may be plated directly onto a culture substrate. The culture substrate may comprise a tissue culture vessel, such as a Petri dish. The vessel may be pre-treated. The cells may be plated onto, and grow on, a gelatinised tissue culture plate.

An example protocol for the gelatin coating of dishes follows. A solution of 0.1% gelatin in distilled water is made and autoclaved. This may be stored at room temp. The bottom of a tissue culture dish is covered with the gelatin solution and incubated for 5-15 min. Remove gelatin and plates are ready to use. Medium should be added before adding cells to prevent hypotonic lysis.

### Serum Free Media

The dissociated or disaggregated embryonic stem cells may be cultured in a medium which may comprise a serum-free medium.

The term “serum-free media” may comprise cell culture media which is free of serum proteins, e.g., fetal calf serum. Serum-free media are known in the art, and are described for example in U.S. Pat. Nos. 5,631,159 and 5,661,034. Serum-free media are commercially available from, for example, Gibco-BRL (Invitrogen).

The serum-free media may be protein free, in that it may lack proteins, hydrolysates, and components of unknown composition. The serum-free media may comprise chemically-defined media in which all components have a known chemical structure. Chemically-defined serum-free media is advantageous as it provides a completely defined system which eliminates variability allows for improved reproducibility and more consistent performance, and decreases possibility of contamination by adventitious agents.

The serum-free media may comprise Knockout DMEM media (Invitrogen-Gibco, Grand Island, N.Y.).

The serum-free media may be supplemented with one or more components, such as serum replacement media, at a concentration of for example, 5%, 10%, 15%, etc. The serum-free media may comprise or be supplemented with 10% serum replacement media from Invitrogen- Gibco (Grand Island, N.Y.).

### Growth Factor

The serum-free medium in which the dissociated or disaggregated embryonic stem cells are cultured may comprise one or more growth factors. A number of growth factors are known in the art, including PDGF, EGF, TGF-a, FGF, NGF, Erythropoietin, TGF-b, IGF-I and IGF-II.

The growth factor may comprise fibroblast growth factor 2 (FGF2). The medium may also contain other growth factors such as platelet-derived growth factor AB (PDGF AB). Both of these growth factors are known in the art. The method may comprise culturing cells in a medium comprising both FGF2 and PDGF AB.

Alternatively, or in addition, the medium may comprise or further comprise epidermal growth factor (EGF). Use of EGF may enhance growth of MSCs. EGF may be used at any suitable concentration, for example 5-10 ng/ml EGF. EGF may be used in place of PDGF. EGF is a protein well known in the art, and is referred to as symbol EGF, Alt. Symbols URG, Entrez 1950, HUGO 3229, OMIM 131530, RefSeq NM_{—}001963, UniProt P01133.

Thus, we disclose the use of media comprising (i) FGF2, (ii) FGF2 and PDGF and (iii) FGF2 and EGF and other combinations.

FGF2 is a wide-spectrum mitogenic, angiogenic, and neurotrophic factor that is expressed at low levels in many tissues and cell types and reaches high concentrations in brain and pituitary. FGF2 has been implicated in a multitude of physiologic and pathologic processes, including limb development, angiogenesis, wound healing, and tumor growth. FGF2 may be obtained commercially, for example from Invitrogen-Gibco (Grand Island, N.Y.).

Platelet Derived Growth Factor (PDGF) is a potent mitogen for a wide range of cell types including fibroblasts, smooth muscle and connective tissue. PDGF, which is composed of a dimer of two chains termed the A chain and B chain, can be present as AA or BB [symbol] consisting of 13.3 kDa A chain and 12.2 B chain. PDGF AB may be obtained commercially, for example from Peprotech (Rocky Hill, N.J.).

The growth factor(s), such as FGF2 and optionally PDGF AB, may be present in the medium at concentrations of about 100 pg/ml, such as about 500 pg/ml, such as about 1 ng/ml, such as about 2 ng/ml, such as about 3 ng/ml, such as about 4 ng/ml, such as about 5 ng/ml. In some embodiments, the medium contains FGF2 at about 5 ng/ml. The medium may also contain PDGF AB, such as at about 5 ng/ml.

### Splitting Cells

Cells in culture will generally continue growing until confluence, when contact inhibition causes cessation of cell division and growth. Such cells may then be dissociated from the substrate or flask, and “split”, subcultured or passaged, by dilution into tissue culture medium and replating.

The methods and compositions described here may therefore comprise passaging, or splitting during culture. The cells in the cell culture may be split at a ratio of 1:2 or more, such as 1:3, such as 1:4, 1:5 or more. The term “passage” designates the process consisting in taking an aliquot of a confluent culture of a cell line, in inoculating into fresh medium, and in culturing the line until confluence or saturation is obtained.

### Selection, Screening or Sorting Step

The method may further comprise a selection or sorting step, to further isolate or select for mesenchymal stem cells.

The selection or sorting step may comprise selecting mesenchymal stem cells (MSC) from the cell culture by means of one or more surface antigen markers. The use of a selection or sorting step further enhances the stringency of sorting and selection specificity for MSCs and furthermore potentially reduces possible contamination from embryonic stem cells such as hESCs and other hESC-derivatives from the starting material. This would then further reduce the risk of teratoma formation and further increase the clinical relevance of the protocol we describe.

A number of methods are known for selection or sorting based on antigen expression, and any of these may be used in the selection or sorting step described here. The selection or [symbol] known in the art, FACS involves exposing cells to a reporter, such as a labelled antibody, which binds to and labels antigens expressed by the cell. Methods of production of antibodies and labelling thereof to form reporters are known in the art, and described for example in Harlow and Lane. The cells are then passed through a FACS machine, which sorts the cells from each other based on the labelling. Alternatively or in addition, magnetic cell sorting (MACS) may be employed to sort the cells.

We have realised that while a number of candidate surface antigens known to be associated with MSCs e.g. CD105, CD73, ANPEP, ITGA4 (CD49d), PDGFRA, some of the MSC associated surface antigens e.g. CD29 and CD49e are also highly expressed in ES cells such as hESCs and their expression are verified by FACS analysis. The association of a surface antigen with MSCs may not be sufficient to qualify the antigen as a selectable marker for isolating MSCs from ES cells such as hESC. Accordingly, the selection or sorting step may employ antigens which are differentially expressed between MSCs and ES cells.

The selection or sorting step of our method may positively select for mesenchymal stem cells based on the expression of antigens. Such antigens may be identified by, for example, comparing the gene expression profiles of hESCs and hESCMSCs.

The selection or sorting step of our method may positively select for mesenchymal stem cells based on the expression of antigens which are identified as expressed on MSCs, but not expressed on ES cells such as hESCs.

CD73 is highly expressed on MSCs, while being not highly expressed on hESCs. Both CD73 and CD105 are highly expressed surface antigens in MSCs and are among the top 20 highly expressed surface antigens in hESC-MSCs relative to hESC, the use of either CD73 or CD105 (or both) as selectable marker for putative MSCs will be equally effective in sorting for putative MSCs generated by differentiating hESCs.

Alternatively, or in addition, the selection or sorting step may negatively select against antigens based on surface antigens that are highly expressed as surface antigen on embryonic stem cells (ES cells) such as hESCs, and not mesenchymal stem cells e.g., hESC-MSC. Selection or sorting may be based on known or previously identified hESC-specific surface antigens such as MI BP, ITGB1BP3 and PODXL, and CD24.

FACS analysis confirms the expression of CD24 on hESC but not hESC-MSCs. Therefore, CD24 may be used as a negative selection or sorting marker either on its own, or in conjunction with CD105 as a positive selectable marker for isolating putative MSCs from differentiating hESC cultures.

### Utility

The methods and compositions described here enable purification of exosomes from secretion of MSCs. The methods and compositions described here allow for fast purification or enrichment of exosomes from bodily fluids for diagnostic assays. The methods and compositions described here allow for the purification of exosomes from bodily fluids and tissue cultures for development of biomarkers.

### Diseases Treatable by Particles From Mesenchymal Stem Cells

Analysis of the proteome of MSCs shows that the proteins expressed are involved in three biological processes: metabolism, defense response, and tissue differentiation including vascularization, hematopoiesis and skeletal development.

Accordingly, the particles from MSCs such as exosomes purified as described here may be used to treat diseases which these functions may have a role in, or whose repair or treatment involves any one or more of these biological processes. Similarly, the proteins expressed by the MSCs, singly or in combination, preferably in the form of particles as described here, may be used to supplement the activity of, or in place of, the MSCs, or media conditioned by the MSCs, for the purpose of for example treating or preventing such diseases.

The gene products expressed by the MSCs are shown to activate important signalling pathways in cardiovascular biology, bone development and hematopoiesis such as Jak-STAT, MAPK, Toll-like receptor, TGF-beta signalling and mTOR signaling pathways. Accordingly, the particles from the MSCs, etc, may be used to prevent or treat a disease in which any of these signalling pathways is involved, or whose aetiology involves one or more defects in any one or more of these signalling pathways.

Accordingly, such particles such as exosomes purified as described here may be used to treat cardiac failure, bone marrow disease, skin disease, burns and degenerative diseases such as diabetes, Alzheimer's disease, Parkinson's disease and cancer.

Such particles such as exosomes purified as described here may also be used to treat myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The particles such as exosomes purified as described here may be used to aid wound healing, scar reduction, bone formation, a bone graft or bone marrow transplantation in an individual.

Unless the context dictates otherwise, the term “conditioned medium” should be taken to include not only cell culture medium exposed to MSCs as well as such a composition comprising one or more, preferably substantially all, the polypeptides which are present in the conditioned medium.

The particles such as exosomes purified as described here may also be used as sources for any of the proteins secreted or expressed by the MSCs. We therefore provide for a method of producing a polypeptide as shown, the method comprising obtaining a particle such as an exosome purified as described here, and isolating the polypeptide from the particle.

### Heart Disease

The mesenchymal stem cell particle such as exosomes purified as described here may be used for treatment or prevention of heart disease.

Heart disease is an umbrella term for a variety for different diseases affecting the heart. As of 2007, it is the leading cause of death in the United States, England, Canada and Wales, killing one person every 34 seconds in the United States alone. Heart disease includes any of the following.

### Coronary Heart Disease

Coronary artery disease is a disease of the artery caused by the accumulation of atheromatous plaques within the walls of the arteries that supply the myocardium. Angina pectoris (chest pain) and myocardial infarction (heart attack) are symptoms of and conditions caused by coronary heart disease. Over 459,000 Americans die of coronary heart disease every year. In the United Kingdom, 101,000 deaths annually are due to coronary heart disease.

### Cardiomyopathy

Cardiomyopathy is the deterioration of the function of the myocardium (i.e., the actual heart muscle) for any reason. People with cardiomyopathy are often at risk of arrhythmia and/or sudden cardiac death. Extrinsic cardiomyopathies—cardiomyopathies where the primary pathology is outside the myocardium itself comprise the majority of cardiomyopathies. By far the most common cause of a cardiomyopathy is ischemia.

The World Health Organization includes as specific cardiomyopathies: Alcoholic cardiomyopathy, coronary artery disease, congenital heart disease, nutritional diseases affecting the heart, ischemic (or ischaemic) cardiomyopathy, hypertensive cardiomyopathy, valvular cardiomyopathy, inflammatory cardiomyopathy.

Also included are:

Cardiomyopathy secondary to a systemic metabolic disease

Intrinsic cardiomyopathies (weakness in the muscle of the heart that is not due to an identifiable external cause)

Dilated cardiomyopathy (DCM, the most common form, and one of the leading indications for heart transplantation. In DCM the heart (especially the left ventricle) is enlarged and the pumping function is diminished)

Hypertrophic cardiomyopathy (HCM or HOCM, a genetic disorder caused by various mutations in genes encoding sarcomeric proteins. In HCM the heart muscle is thickened, which can obstruct blood flow and prevent the heart from functioning properly),

Arrhythmogenic right ventricular cardiomyopathy (ARVC, which arises from an electrical disturbance of the heart in which heart muscle is replaced by fibrous scar tissue. The right ventricle is generally most affected)

Restrictive cardiomyopathy (RCM, which is the least common cardiomyopathy. The walls of the ventricles are stiff, but may not be thickened, and resist the normal filling of the heart with blood).

Noncompaction Cardiomyopathy—the left ventricle wall has failed to properly grow from birth and such has a spongy appearance when viewed during an echocardiogram.

### Cardiovascular Disease

Cardiovascular disease is any of a number of specific diseases that affect the heart itself and/or the blood vessel system, especially the veins and arteries leading to and from the heart. Research on disease dimorphism suggests that women who suffer with cardiovascular disease usually suffer from forms that affect the blood vessels while men usually suffer from forms that affect the heart muscle itself. Known or associated causes of cardiovascular disease include diabetes mellitus, hypertension, hyperhomocysteinemia and hypercholesterolemia.

Types of cardiovascular disease include atherosclerosis

### Ischaemic Heart Disease

Ischaemic heart disease is disease of the heart itself, characterized by reduced blood supply to the organs. This occurs when the arteries that supply the oxygen and the nutrients gets stopped and the heart will not get enough of the oxygen and the nutrients and will eventually stop beating.

### Heart Failure

Heart failure, also called congestive heart failure (or CHF), and congestive cardiac failure (CCF), is a condition that can result from any structural or functional cardiac disorder that impairs the ability of the heart to fill with or pump a sufficient amount of blood throughout the body. Cor pulmonale is a failure of the right side of the heart.

### Hypertensive Heart Disease

Hypertensive heart disease is heart disease caused by high blood pressure, especially localised high blood pressure. Conditions that can be caused by hypertensive heart disease include: left ventricular hypertrophy, coronary heart disease, (Congestive) heart failure, hypertensive cardiomyopathy, cardiac arrhythmias, inflammatory heart disease, etc.

Inflammatory heart disease involves inflammation of the heart muscle and/or the tissue surrounding it. Endocarditis comprises inflammation of the inner layer of the heart, the endocardium. The most common structures involved are the heart valves. Inflammatory cardiomegaly. Myocarditis comprises inflammation of the myocardium, the muscular part of

[symbol]

### Valvular Heart Disease

Valvular heart disease is disease process that affects one or more valves of the heart. The valves in the right side of the heart are the tricuspid valve and the pulmonic valve. The valves in the left side of the heart are the mitral valve and the aortic valve. Included are aortic valve stenosis, mitral valve prolapse and valvular cardiomyopathy.

[The above text is adapted from Heart disease. (2009 Feb. 3). In Wikipedia, The Free Encyclopedia. Retrieved 06:33, Feb. 20, 2009, from http://en.wikipedia.org/w/index.php?title=Heart_disease&oldid=268290924]

### Delivery of Particles

The particles such as exosomes purified as described in this document may be delivered to the human or animal body by any suitable means.

We therefore describe a delivery system for delivering a particles such as exosomes purified as described in this document to a target cell, tissue, organ, animal body or human body, and methods for using the delivery system to deliver particles to a target.

The delivery system may comprise a source of particles such as exosomes purified as described here, such as a container containing the particles. The delivery system may comprise a dispenser for dispensing the particles to a target.

Accordingly, we provide a delivery system for delivering a particle such as an exosome purified as described here, comprising a source of particles as described in this document together with a dispenser operable to deliver the particles to a target.

We further provide for the use of such a delivery system in a method of delivering a particles to a target.

Delivery systems for delivering fluid into the body are known in the art, and include injection, surgical drips, cathethers (including perfusion cathethers) such as those described in U.S. Pat. No. 6,139,524, for example, drug delivery catheters such as those described in U.S. Pat. No. 7,122,019.

Delivery to the lungs or nasal passages, including intranasal delivery, may be achieved using for example a nasal spray, puffer, inhaler, etc as known in the art (for example as shown

[symbol]

Delivery to the kidneys may be achieved using an intra-aortic renal delivery catheter, such as that described in U.S. Pat. No. 7,241,273.

It will be evident that the particular delivery should be configurable to deliver the required amount of particles at the appropriate interval, in order to achieve optimal treatment.

The particles such as exosomes purified as described here may for example be used for the treatment or prevention of atherosclerosis. Here, perfusion of particles may be done intravenously to stabilize atherosclerotic plaques or reduce inflammation in the plaques. The particles may be used for the treatment or prevention of septic shock by intravenous perfusion.

The particles such as exosomes purified as described here may be used for the treatment or prevention of heart failure. This may be achieved by chronic intracoronary or intramyocardially perfusion of particles to retard remodeling or retard heart failure. The particles may be used for the treatment or prevention of lung inflammation by intranasal delivery.

The particles may be used for the treatment or prevention of dermatological conditions e.g. psoriasis. Long term delivery of particles may be employed using transdermal microinjection needles until the condition is resolved.

It will be evident that the delivery method will depend on the particular organ to which the particles is to be delivered, and the skilled person will be able to determine which means to employ accordingly.

As an example, in the treatment of cardiac inflammation, the particles may be delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen.

Any variety of coronary catheter, or a perfusion catheter, may be used to administer the compound. Alternatively the particles may be coated or impregnated on a stent that is placed in a coronary vessel.

### Tissue Regeneration

Mesenchymal stem cells particles isolated according to the methods and compositions described here may also be used for tissue reconstitution or regeneration in a human patient in need thereof. The particles such as exosomes may be administered in a manner that permits them to graft to the intended tissue site and reconstitute or regenerate the functionally deficient area.

For example, the methods and compositions described here may be used to modulate the differentiation of stem cells. Mesenchymal stem cell particles such as exosomes purified as described here therefrom may be used for tissue engineering, such as for the growing of skin grafts. Modulation of stem cell differentiation may be used for the bioengineering of artificial organs or tissues, or for prosthetics, such as stents.

### Cancer

Mesenchymal stem cell particles such as exosomes purified as described here may be used for the treatment of cancer.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia.

More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer. Further examples are solid tumor cancer including colon cancer, breast cancer, lung cancer and prostrate cancer, hematopoietic malignancies including leukemias and lymphomas, Hodgkin's disease, aplastic anemia, skin cancer and familiar adenomatous polyposis. Further examples include brain neoplasms, colorectal neoplasms, breast neoplasms, cervix neoplasms, eye neoplasms, liver neoplasms, lung neoplasms, pancreatic neoplasms, ovarian neoplasms, prostatic neoplasms, skin neoplasms, testicular neoplasms, neoplasms, bone neoplasms, trophoblastic neoplasms, fallopian tube neoplasms, rectal neoplasms, colonic neoplasms, cancer, pancreatic cancer, colorectal cancer, lung cancer, malignant melanoma, leukaemia, lympyhoma, ovarian cancer, cervical cancer and biliary tract carcinoma are also included.

The mesenchymal stem cell particles such as exosomes purified as described here may also be used in combination with anticancer agents such as endostatin and angiostatin or cytotoxic agents or chemotherapeutic agent. For example, drugs such as such as adriamycin, daunomycin, cis-platinum, etoposide, taxol, taxotere and alkaloids, such as vincristine, and antimetabolites such as methotrexate. The term “cytotoxic agent” as used herein refers to a substance that inhibits or prevents the function of cells and/or causes destruction of cells. The term is intended to include radioactive isotopes (e.g. I, Y, Pr), chemotherapeutic agents, and toxins such as enzymatically active toxins of bacterial, fungal, plant or animal origin, or fragments thereof.

Also, the term includes oncogene product/tyrosine kinase inhibitors, such as the bicyclic ansamycins disclosed in WO 94/22867; 1,2-bis(arylamino) benzoic acid derivatives disclosed in EP 600832; 6,7-diamino-phthalazin-1-one derivatives disclosed in EP 600831; 4,5-bis(arylamino)-phthalimide derivatives as disclosed in EP 516598; or peptides which inhibit binding of a tyrosine kinase to a SH2-containing substrate protein (see WO 94/07913, for example). A “chemotherapeutic agent” is a chemical compound useful in the treatment of cancer. Examples of chemotherapeutic agents include Adriamycin, Doxorubicin, 5-Fluorouracil (5-FU), Cytosine arabinoside (Ara-C), Cyclophosphamide, Thiotepa, Busulfan, Cytoxin, Taxol, Methotrexate, Cisplatin, Melphalan, Vinblastine, Bleomycin, Etoposide, Ifosfamide, Mitomycin C, Mitoxantrone, Vincristine, VP-16, Vinorelbine, Carboplatin, Teniposide, Daunomycin, Carminomycin, Aminopterin, Dactinomycin, Mitomycins, Nicotinamide, Esperamicins (see U.S. Pat. No. 4,675,187), Melphalan and other related nitrogen mustards, and endocrine therapies (such as diethylstilbestrol (DES), Tamoxifen, LHRH antagonizing drugs, progestins, anti-progestins etc).

### EXAMPLES

Exosomes are bilipid membrane vesicles with proteins embedded in the membranes. It has been previously demonstrated that brain tumor exosomes have a alkaline pI (isoelectric point) of >8.1[1].

We have previously demonstrated that culture medium conditioned by MSCs derived purified as a population of homogenously sized particles by size exclusion high performance liquid chromatograhy (HPLC) [2,3].

### Example 0

### Example Anion Exchange Chromatography

Ion exchange chromatography may be performed as described herein. A anion exchange resin may be employed.

The anion exchange resin may be prepared according to known methods, following the manufacturer's instructions. An equilibration buffer may be passed through the ion exchange resin prior to loading the composition comprising the mesenchymal stem cell particle (such as exosome) and one or more contaminants onto the resin. The equilibration buffer may be the same as the loading buffer, but this is not required.

Following equilibration, an aqueous solution comprising the mesenchymal stem cell particle (such as exosome) and contaminant(s) may be loaded onto the anion exchange resin using a buffer that is at a pH and/or conductivity such that the mesenchymal stem cell particle (such as exosome) and the contaminant bind to the anion exchange resin. As discussed above, the equilibration buffer may be used for loading.

The amount of the mesenchymal stem cell particle (such as exosome) loaded onto the resin may depend on a variety of factors, including, for example, the capacity of the resin, the desired yield, and the desired purity. For example, from about 1 mg of protein/ml of resin to about 100 mg of protein/ml of resin, such as from about 10 mg/ml to about 75 mg/ml such as from about 15 mg/ml to about 45 mg/ml of the mesenchymal stem cell particle (such as exosome) may be loaded on the ion exchange resin.

After loading, the anion exchange resin may be washed. During the wash process, wash buffer is passed over the resin. The composition of the wash buffer may be typically chosen to elute as many contaminants as possible from the resin without eluting a substantial amount of the mesenchymal stem cell particle (such as exosome). This may be achieved by using a wash buffer with an increased conductivity or pH, or both, compared to the equilibration buffer. The composition of the was buffer may be constant or variable over the wash process.

The wash buffer may comprise equilibration buffer in which the salt concentration has example, the wash buffer may comprise a mixture of equilibration buffer and elution buffer. In this case, the desired salt concentration in the wash buffer may be achieved by increasing the percentage of the higher salt buffer in the wash buffer. The elution buffer typically has a higher salt concentration and conductivity than the equilibration buffer. For example, the elution buffer may have a conductivity of between about 8 mS/cm and about 10 mS/cm, such as between about 8.5 mS/cm and 9.5 mS/cm, while the equilibration buffer has a conductivity of between about 4 and 6 mS/cm, such as between about 4.5 and 5.5 mS/cm. Thus, as the percentage of elution buffer is increased, the salt concentration and the conductivity of the wash buffer increase.

The desired mesenchymal stem cell particle (such as exosome) may be subsequently eluted from the ion exchange resin. This may be achieved using an elution buffer that has a pH and/or conductivity such that the desired mesenchymal stem cell particle (such as exosome) no longer binds to the ion exchange resin and therefore is eluted therefrom. Thus, the conductivity of the elution buffer may be such that it exceeds that of the equilibration buffer. Alternatively, or in addition, the pH of the elution buffer may be increased relative to the equilibration buffer (for example, the pH of the elution buffer may about 6.0). The change in conductivity and/or pH from the wash buffer to the elution buffer may be step-wise or gradual, as desired. As discussed above, the elution buffer may have a conductivity of between about 8 mS/cm and about 10 mS/cm, such as between about 8.5 mS/cm and 9.5 mS/cm. Hence, the mesenchymal stem cell particle (such as exosome) may be retrieved from the anion exchange resin at this stage in the method.

As an example, a single parameter (i.e. either conductivity or pH) may be changed to achieve elution of both the mesenchymal stem cell particle (such as exosome) and contaminant, while the other parameter (i.e. pH or conductivity, respectively) remains about constant. For example, while the conductivity of the various buffers may differ, the pH's thereof may be essentially the same.

The ion exchange resin may be regenerated with a regeneration buffer after elution of the mesenchymal stem cell particle (such as exosome), such that the column can be re-used. Generally, the conductivity and/or pH of the regeneration buffer may be such that substantially all contaminants and the mesenchymal stem cell particles (such as exosome) are eluted from the ion exchange resin. Generally, the regeneration buffer has a very high conductivity for eluting contaminants and mesenchymal stem cell particles (such as exosome) from the ion exchange resin.

### Example 1

### Materials and Methods: Fractionation of Conditioned Medium by Anion Exchange Chromatography

Conditioned medium (CM) was fractionated by Ion Exchange Chromatography using a commercially available spin column, Pierce Strong Anion Exchange Spin Columns (Thermo Fisher Scientific Inc., Rockford, Ill.).

Briefly, the column was first equilibrated with 20 mM Tris-HCL Buffer pH 8.8. 60 μg CM in 100 μL PBS was then loaded onto the column, spun at 2000×g for 5 mins. The column was then washed sequentially with 100 μL of 20 mM Tris-HCl Buffer pH 8.8 containing increasing NaCl concentration starting with 500 μM, 1 mM, 2 mM, 4 mM, 8 mM, 16 mM, 32 mM, 62.5 mM, 125 mM, 250 mM, 500 mM, 1M and 2M NaCl.

Each eluting fraction was collected and 10 μL was resolved on 4-12% SDS-polyacrylamide gels. The gels were either stained or electroblotted onto a nitrocellulose membrane.

The membrane was probed with either 1:200 dilution of mouse anti-20S proteasome antibody that recognises the alpha subunits or 1:50 dilution of mouse anti-human CD9 antibody.

The secondary antibody was 1:1250 of HRP conjugated donkey anti-mouse IgG antibody. All antibodies were purchased from Santa Cruz.

The bound antibodies were visualized using HRP-enhanced chemiluminescent substrate (Thermo Fisher Scientific Inc., Waltham, Mass.) and exposure to an X-ray film. Silver stain was done using a commercially available SilverQuestTM Silver Staining Kit (Invitrogen, Carlsbad, Calif.).

### Example 2

### Results: Fractionation of Conditioned Medium by Anion Exchange Chromatography

The aim of this Example is to determine if exosomes in this MSC conditioned medium (CM) were charged and could also be purified rapidly by ion exchange chromatography using

[symbol]

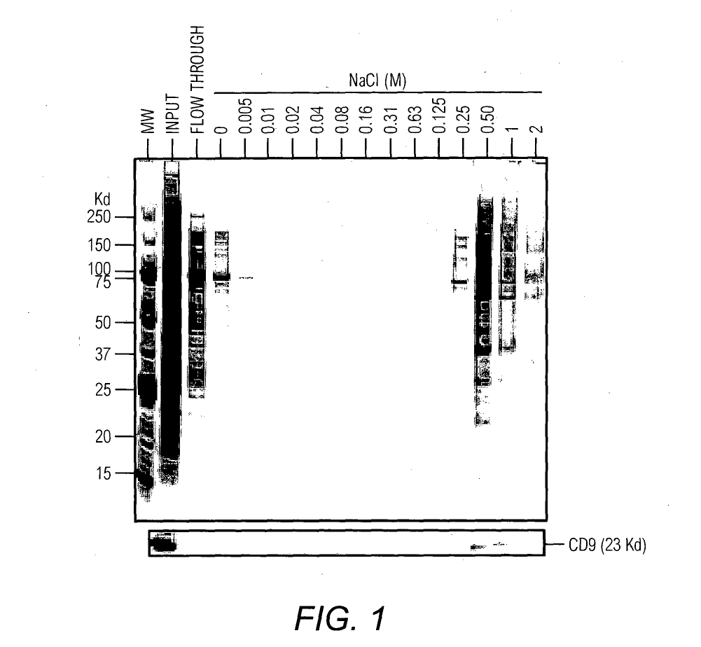

We fractionated the CM first by anion exchange chromatography (FIG. 1). Exosomes in the CM was monitored by the presence of CD9, a surface protein commonly associated with exosomes and found in exosomes secreted by MSCs [2,3].

As shown in FIG. 1, CD9 was found to be bound by the anion resins at pH8.8 and were eluted at high NaCl concentration of 0.5-2M showing that MSC exosomes were negatively charged and have a lower or more acidic pI than the very basic pI observed for brain tumor exosomes as previously reported [1].

### Example 3

### Materials and Methods: Fractionation of Conditioned Medium by Cation Exchange Chromatography

Conditioned medium (CM) was fractionated by Ion Exchange Chromatography using a commercially available spin column Pierce Strong Cation Exchange Spin Columns (Thermo Fisher Scientific Inc., Rockford, Ill.).

Briefly, the column was first equilibrated with 100 μL 20 mM sodium acetate buffer, pH 5.5. 60 μg CM in 100 μL PBS was then loaded onto the column, spun at 2000×g for 5 mins. The column was then washed sequentially with 100 μL of 20 mM sodium acetate buffer pH 5.5 containing increasing NaCl concentration starting with 500 μM, 1 mM, 2 mM, 4 mM, 8 mM, 16 mM, 32 mM, 62.5 mM, 125 mM, 250 mM, 500 mM, 1M and 2M NaCl.

Each eluting fraction was collected and 10 μL was resolved on 4-12% SDS-polyacrylamide gels. The gels were either stained or electroblotted onto a nitrocellulose membrane.

The membrane was probed with either 1:200 dilution of mouse anti-20S proteasome antibody that recognises the alpha subunits or 1:50 dilution of mouse anti-human CD9 antibody.

The secondary antibody was 1:1250 of HRP-conjugated donkey anti-mouse IgG antibody. All antibodies were purchased from Santa Cruz.

The bound antibodies were visualized using HRP-enhanced chemiluminescent substrate (Thermo Fisher Scientific Inc., Waltham, MA) and exposure to an X-ray film. Silver stain was done using a commercially available SilverQuest™ Silver Staining Kit (Invitrogen,

[symbol]

### Example 4

### Results: Fractionation of Conditioned Medium by Cation Exchange Chromatography

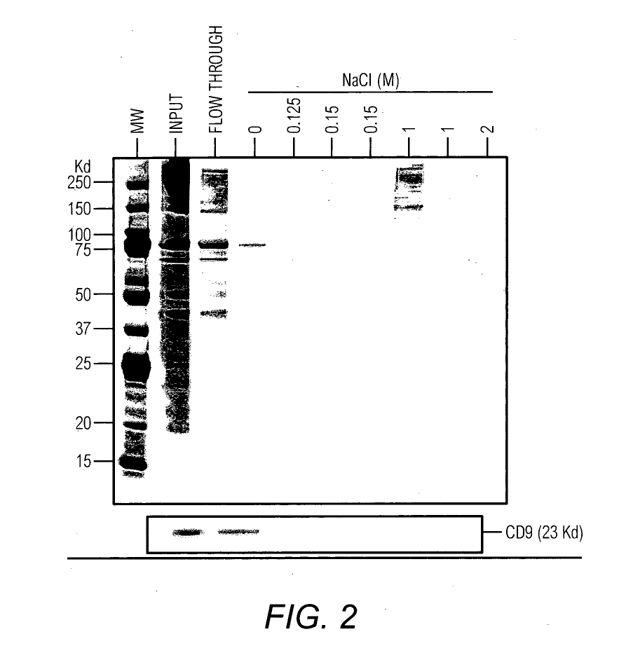

Consistent with the results from Example 1, fractionation of the CM by cation exchange chromatography at pH5.5 revealed that CD9 was present in the flow-through and was not bound by the resin (FIG. 2).

### Example 5

### Conclusions

1. The pI of exosomes secreted by MSCs is acidic in contrast to basic pI of tumor exosomes [1]. Therefore, this shows for the first time that different types of exosomes could have different pIs.

2. This difference in pI provides a method to distinguish or/and purify exosomes on the basis of their charges.

3. This technology could be used to purify or enriched for different types of exosomes from different biological fluids or cell-conditioned medium for therapeutic and diagnostic purposes.

### REFERENCES

1. Graner MW, Alzate O, Dechkovskaia A M, Keene J D, Samson J H, et al. (2009) Proteomic and immunologic analyses of brain tumor exosomes. Faseb J 23: 1541-1557.

2. Lai R C, Arslan F, Lee M M, Sze NS, Choo A, et al. (2010) Exosome secreted by MSC reduces myocardial ischemia/reperfusion injury. Stem Cell Res 4: 214-222.

3. Lai R C, Arslan F, Tan S S, Tan B, Choo A, et al. (2010) Derivation and characterization of human fetal MSCs: An alternative cell source for large-scale production of cardioprotective microparticles. J Mol Cell Cardiol 48: 1215-1224.

Pan, B. T. & Johnstone, R. M. Fate of the transferrin receptor during maturation of sheep reticulocytes in vitro: selective externalization of the receptor. Cell 33, 967-978 (1983).

Thery, C., Ostrowski, M. & Segura, E. Membrane vesicles as conveyors of immune responses. Nat Rev Immunol 9, 581-593 (2009).

Fevrier, B. & Raposo, G. Exosomes: endosomal-derived vesicles shipping

[symbol]

Keller, S., Sanderson, M. P., Stoeck, A. & Altevogt, P. Exosomes: from biogenesis and secretion to biological function. Immunol Lett 107, 102-108 (2006).

Zitvogel, L., et al. Eradication of established murine tumors using a novel cell-free vaccine: Dendritic cell-derived exosomes. Nature Medicine 4, 594-600 (1998).

Wolfers, J., et al. Tumor-derived exosomes are a source of shared tumor rejection antigens for CTL cross-priming. Nature Medicine 7, 297-303 (2001).

Skokos, D., et al. Mast cell-derived exosomes induce phenotypic and functional maturation of dendritic cells and elicit specific immune responses in vivo. Journal of Immunology 170, 3037-3045 (2003).

Taylor, D. D. & Gercel-Taylor, C. Tumour-derived exosomes and their role in cancer-associated T-cell signalling defects. British Journal of Cancer 92, 305-311 (2005).

Lai, R. C., et al. Exosome secreted by MSC reduces myocardial ischemia/reperfusion injury. Stem Cell Res 4, 214-222 (2010).

Lai, R. C., et al. Derivation and characterization of human fetal MSCs: an alternative cell source for large-scale production of cardioprotective microparticles. J Mol Cell Cardiol 48, 1215-1224 (2010).

Sze, S.K., et al. Elucidating the secretion proteome of human embryonic stem cell-derived mesenchymal stem cells. Mol Cell Proteomics 6, 1680-1689 (2007).

Chen, T. S., et al. Mesenchymal stem cell secretes microparticles enriched in pre-microRNAs. Nucleic Acids Res 38, 215-224 (2010).

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby

[symbol]

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of purifying a mesenchymal stem cell exosome particle, the method comprising separating the mesenchymal stem cell exosome particle on the basis of its negative charge.

2. The method according to claim 1, which comprises:
(a) providing a composition comprising mesenchymal stem cell exosome particles;
(b) applying the composition comprising mesenchymal stem cell exosome particles to an ion exchange resin to enable mesenchymal stem cell particles to bind to the ion exchange resin; and
(c) eluting bound mesenchymal stem cell exosome particles.

3. The method according to claim 2, in which the composition comprising mesenchymal stem cell exosome particles is applied to the ion exchange resin in a spin column.

4. The method according to claim 3, in which the composition comprising mesenchymal stem cell exosome particles is applied to the ion exchange spin column at an alkaline pH.

5. The method according to claim 2, in which mesenchymal stem cell particles are eluted at a salt concentration of 500 μM or more.

6. The method according to claim 2, which comprises detecting the alpha subunit of the 20S proteasome or CD9, or both, in fractions of the eluate of step (c) to detect mesenchymal stem cell exosome particles.

7. The method according to claim 1, in which the mesenchymal stem cell exosome particle comprises at least one biological property of a mesenchymal stem cell conditioned medium.

8. The method according to claim 1, in which the mesenchymal stem cell exosome particle:
(a) reduces infarct size in a mouse or pig model of myocardial ischemia and reperfusion injury;
(b) reduces oxidative stress in an in vitro assay of hydrogen peroxide (H₂O₂)-induced cell death;
(c) comprises a vesicle;
(d) comprises a complex of molecular weight >100 kDa;
(e) comprises a complex of molecular weight >300 kDa;
(f) comprises a complex of molecular weight >1000 kDa;
(g) has a size of between 2 nm and 200 nm;
(h) has a hydrodynamic radius of below 100 nm;
(i) comprises a lipid selected from the group consisting of: phospholipid, phosphatidyl serine, phosphatidyl inositol, phosphatidyl choline, shingomyelin, ceramides, glycolipid, cerebroside, steroids, and cholesterol;
(j) comprises a lipid raft;
(k) is insoluble in non-ionic detergent;
(l) is such that proteins of the molecular weights specified in (d), (e) or (f) substantially remain in the complexes of the molecular weights specified in those claims, when the particle is treated with a non-ionic detergent;
(m) is sensitive to cyclodextrin;
(n) comprises ribonucleic acid (RNA; or
(o) comprises a surface antigen selected from the group consisting of: CD9, CD109 and thy-1.

9. A method of producing a mesenchymal stem cell particle, the method comprising separating the particle from other components of a mesenchymal stem cell conditioned medium (MSC-CM) composition based on charge.

10. The method according to claim 9, in which the method comprises anion exchange chromatography.

11. The method according to claim 9, in which the method comprises the use of an anion exchange spin column.

12. The method of claim 6, wherein the alpha subunit of the 20S proteasome is detected by an anti-20S proteasome antibody that recognises the alpha subunit or in which CD9 is detected by an anti-CD9 antibody, or both.

13. The method of claim 7, in which the at least one biological property of the mesenchymal stem cell conditioned medium (MSC-CM) is cardioprotection.

14. The method of claim 8, wherein the exosome comprises at least 70% of proteins in a mesenchymal stem cell conditioned medium (MSC-CM).

15. The method of claim 8, wherein the size of the mesenchymal stem cell particle is determined by filtration against a 0.2 μM filter and concentration against a membrane with a molecular weight cut-off of 10 kDa.

16. The method of claim 8, wherein the size of the mesenchymal stem cell particle is determined by electron microscopy.

17. The method of claim 8, wherein the hydrodynamic radius is determined by laser diffraction or dynamic light scattering.

18. The method of claim 8, wherein the non-ionic detergent is Triton-X100.

19. The method of claim 9, wherein the mesenchymal stem cell particle is an exosome.
